(19) United States
(12) Patent Application Publication
(10) Pub. No.: US 2024/0358491 A1
(43) Pub. Date: Oct. 31, 2024

(54) TISSUE SCAFFOLD IMPLANT DEVICES FOR STENOTIC LUMENS

(71) Applicants: THE REGENTS OF THE UNIVERSITY OF MICHIGAN, Ann Arbor, MI (US); Georgia Tech Research Corporation, Atlanta, GA (US)

(72) Inventors: Scott J. HOLLISTER, Atlanta, GA (US); David A. ZOPF, Dexter, MI (US)

(73) Assignees: THE REGENTS OF THE UNIVERSITY OF MICHIGAN, Ann Arbor, MI (US); Georgia Tech Research Corporation, Atlanta, GA (US)

(21) Appl. No.: 18/687,204

(22) PCT Filed: Aug. 31, 2022
(86) PCT No.: PCT/US2022/042167

§ 371 Date: Feb. 27, 2024

Related U.S. Application Data
(60) Provisional application No. 63/239,004, filed on Aug. 31, 2021.

(51) Int. Cl. A61F 2/04

(52) U.S. Cl. CPC A61F 2/04; A61F 2002/046; A61F 2210/0009; A61F 2210/0014; A61F 2210/0066; A61F 2210/0071; A61F 2220/0016; A61F 2230/0019; A61F 2230/0026; A61F 2230/0052; A61F 2230/0089

(57) ABSTRACT

A tissue scaffold implant for expanding a stenotic lumen is provided that includes a bridge structure defining at least two angled sides and a lateral anchor integrally formed with the bridge structure. The lateral anchor is configured to be disposed against at least a portion of an external circumference of the stenotic lumen in a region near an opening formed in the stenotic lumen. At least two seat regions are defined between the at least two angled sides of the bridge structure and the lateral anchor. The at least two seat regions are configured to be received within and support the opening within the stenotic lumen, and the bridge structure and lateral anchor comprise a bioacceptable material.

### CROSS-REFERENCE TO RELATED APPLICATIONS

This application claims the benefit of U.S. Provisional Application No. 63/239,004, filed on Aug. 31, 2021. The entire disclosure of the above application is incorporated herein by reference.

### GOVERNMENT SUPPORT

This invention was made with government support under HD086201 awarded by the National Institutes of Health. The government has certain rights in the invention.

### FIELD

The present disclosure relates to a tissue scaffold implant for expanding a stenotic lumen, such as a stenotic trachea or glottis.

### BACKGROUND

This section provides background information related to the present disclosure which is not necessarily prior art.

Various lumens in the human body may suffer from stenosis. For example, airway stenosis is a narrowing of an airway that can be caused from 1) scarring acquired from prolonged periods of intubation and ventilation; 2) congenital birth defects; 3) tumors; 4) autoimmune disorders, 5) infection, or 6) airway injury, by way of example. The effect of airway stenosis include difficulty breathing (can be severe), noisy breathing, excessive coughing, frequent pneumonia, apnea, and death.

One method of current treatment is balloon dilation, which is a short-term solution. A second method is laryngotracheal reconstruction that involves inserting a piece of the patient's cartilage into the narrowed section of the trachea in order to widen it, for example, in an anterior or posterior fashion. An endoscopic posterior cricoid split with rib graft (EPCS/RG) was introduced in 2002 by Dr. Andrew Inglis for the correction of posterior glottic stenosis (PGS) and subglottic stenosis (SGS). The procedure, by expanding a posterior cricoid, has been effective for decannulation in pediatric patients, with particular efficacy for PGS and showing statistical improvements in decannulation rates in SGS and bilateral vocal fold immobility (BVFI). EPCS/RG has also shown early promise in its translation to adult patients. Currently, the EPCS/RG requires the harvesting of costal cartilage, which is then carved into a desired shape prior to implantation. The cartilage harvest has rare, but critical intraoperative complications including pneumothorax/hemothorax during the rib harvesting and post-operative site infection amongst others. The carving of the cartilage graft itself requires technical expertise with little tolerance of technical error. If the harvested cartilage is carved improperly or damaged during implantation, the surgeon's only option may be returning to the harvest site for additional cartilage. Thus, limiting aspects of the current procedures include donor site morbidity, significant donor site pain, resultant splinting, hypoventilation, atelectasis, and pneumonia. Pneumothorax, or pleural injury, can occur at the time of harvest with resultant significant morbidity and potential for mortality. Donor site infection, hematoma, seroma, or dehiscence are additional risks. Each of these occurrences can lengthen inpatient hospital stay and result in increased health care resources and costs.

Furthermore, the rib carving and harvest demand significant resources including surgeon, anesthetic, and operating room time. The complex geometric carving requires robust experience with little room for error. Significant surgical training and expertise are required to perform the procedure, limiting availability to offer this procedure as an option and often defaulting to tracheostomy as treatment. Tracheostomy, particularly in pediatric population, carries significant morbidity and mortality and tremendously affects patient and family quality of life.

A prefabricated, tissue engineered graft scaffold would allow a transformative approach to such stenotic widening procedures. With current surgical techniques, the airway surgeon estimates the width of the rib graft and has a single chance to carve an optimal geometry. There is no ability to gauge if alternative width grafts would be better suited for a given application. A tissue graft scaffold—with predefined sizing grafts—allows for optimizing the graft fit. Further, mock graft/implants of varying widths further allow for an optimization process. That size scaffold could then be selected—all with the avoidance of second surgical site donor harvest.

### SUMMARY

This section provides a general summary of the disclosure, and is not a comprehensive disclosure of its full scope or all of its features.

In certain aspects, the present disclosure relates to a tissue scaffold implant for expanding a stenotic lumen. The tissue scaffold implant may comprise a bridge structure defining at least two angled sides. The tissue scaffold implant may also comprise a lateral anchor integrally formed with the bridge structure, where the lateral anchor is configured to be disposed against at least a portion of an external circumference of the stenotic lumen in a region near an opening formed in the stenotic lumen. The tissue scaffold implant further comprises at least two seat regions defined between the at least two angled sides of the bridge structure and the lateral anchor. The at least two seat regions are configured to be received within and support the opening within the stenotic lumen. Further, the bridge structure and lateral anchor comprise a bioacceptable material.

In certain aspects, the bridge structure comprises two wings defining the at least two angled sides and a concave junction is respectively defined between the lateral anchor and each of the two wings to define the at least two seat regions.

In certain aspects, an angle defined between each of the at least two angled sides of the bridge structure and the lateral anchor is less than or equal to about 120°.

In certain aspects, the bridge structure defines a trapezoidal pyramidal shape.

In certain aspects, the lateral anchor defines a solid body having at least one hollow central region.

In certain aspects, the lateral anchor defines a plurality of struts in a central region.

In certain aspects, the bridge structure comprises at least two solid walls defining each of the angled sides.

In certain aspects, the at least two angled sides each comprise a plurality of angled support teeth spaced apart from one another.

In certain aspects, the lateral anchor defines a curved sleeve that conforms to at least a portion of the exterior circumference of the stenotic lumen.

In certain aspects, the lateral anchor defines a rectangular shape.

In certain aspects, the lateral anchor defines a polygonal shape.

In certain aspects, the lateral anchor defines a truncated diamond shape and the bridge structure defines at least two wings that each define at least two angled sides complementary with the polygonal shape.

In certain aspects, the lateral anchor defines a solid body having an aperture defined therein configured to receive a cylindrical tissue graft.

In certain aspects, the tissue graft comprises an upper region of perichondrium and a lower region of cartilage.

In certain aspects, a cross-sectional shape of the implant is a K-shape.

In certain aspects, the at least two angled sides each comprise a surface comprising at least one securement feature.

In certain further aspects, the at least one securement feature comprises a plurality of angled support teeth spaced apart from one another.

In certain further aspects, the at least one securement features is a textured surface, so that the at least two angled sides comprise the textured surface.

In certain aspects, the bioacceptable material is selected from the group consisting of: polycaprolactone (PCL), polyethylene glycol (PEG), polylactic acid (PLA), polyurethane (PU), polyglycerol dodecanedioate (PGD), extracellular tissue matrix, polysiloxane, nickel-titanium alloy (nitinol), polyetheretherketone (PEEK), polyetherketoneketone (PEKK), and combinations thereof.

In certain aspects, the tissue scaffold implant further comprises at least one bioactive material.

In certain aspects, the tissue scaffold implant further comprises one or more openings configured to receive a needle for creating a suture between the tissue scaffold implant and the stenotic lumen.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C show an exemplary procedure for placing a tissue scaffold implant device according to certain aspects of the present disclosure. FIG. 1A shows a surgical incision formed in a stenotic lumen in the form of a trachea and cricoid portions of an airway. FIG. 1B shows the tissue scaffold implant device in the opening made by the incision. FIG. 1C is a cross-sectional view where the tissue scaffold implant device has been implanted for enlarging a dimeter of the stenotic lumen.

FIGS. 2A-2B show two variations of a tissue scaffold implant device according to certain aspects of the present disclosure. FIG. 2A shows a first tissue scaffold implant device having a solid bridge support in the form an inverted truncated trapezoidal wedge. FIG. 2B shows a second tissue scaffold implant device having a bridge support in the form an inverted truncated trapezoidal wedge with a plurality of apertures formed therein configured to receive tissue grafts to facilitate ingrowth of tissue after implantation.

FIGS. 3A-3I show another variation of a tissue scaffold implant device according to certain aspects of the present disclosure having a fillet or trussed structure that defines a “K-shape.” FIG. 3A shows a front perspective view of the tissue scaffold implant device. FIG. 3B shows a front view, FIG. 3C shows a side view, FIG. 3D shows a cross-sectional view, and FIG. 3E shows another perspective view of the tissue scaffold implant device. FIG. 3F shows a drawing of a side view, FIG. 3G shows a drawing of a front view, FIG. 3H shows a perspective drawing, and FIG. 3I shows a cross-sectional drawing of the tissue scaffold implant device.

FIGS. 4A-4C show yet another variation of a tissue scaffold implant device according to certain aspects of the present disclosure that defines a “K-shape” and has a holder for a tissue insert, which may include perichondrium and cartilage. FIG. 4A is a perspective view of the tissue scaffold implant device prior to introduction of any tissue insert/graft. FIG. 4B shows the tissue insert/graft being placed into the holder of the tissue scaffold implant device. FIG. 4C shows the tissue insert/graft being fully secured and placed into the holder for facilitating tissue ingrowth during implantation.

FIGS. 5A-5C shows multiple tissue scaffold implant devices having a “K-shape” and trussed structures. FIG. 5A shows multiple tissue scaffold implant devices with a variety of dimensions formed according to certain aspects of the present disclosure. FIG. 5B shows one variation of a tissue scaffold implant formed according to certain aspects of the present disclosure having a “K-shape” with longitudinal supports across the angled sides of the bridge portion to help minimize or prevent inward collapse, along with the angled sides defined by walls having at least one opening. FIG. 5C shows another variation of a tissue scaffold implant formed according to certain aspects of the present disclosure having a “K-shape” longitudinal supports across the angled sides of the bridge portion to help minimize or prevent inward collapse and outward facing lateral walls of the angled sides comprising securement features in the form of angled teeth.

FIGS. 6A-6B show two different views of yet another variation of a tissue scaffold implant prepared according to certain aspects of the present disclosure having a bridge structure in the form an inverted truncated trapezoidal wedge and a lateral anchor in the form of a partial circumferential sleeve that can be placed around an external circumference of a stenotic lumen. FIG. 6A is a perspective top view, while FIG. 6B is a perspective side view.

FIGS. 7A-7B show two views of another variation of a tissue scaffold implant prepared according to certain aspects of the present disclosure having an angled polygonal shape in a form of a truncated diamond so that is complementary to a shape of surgically formed opening in a stenotic lumen. FIG. 7A is a side perspective view taken from a first angle, while FIG. 7B is a perspective side view taken from a second angle.

FIGS. 8A-8F show pictures taken from gross dissection of posterior cricoid and 3D printed implants prepared in accordance with certain variations of the present disclosure and implanted in three pigs. FIGS. 8A-8C show inspection of the implantation sites post mortem.

FIGS. 8D-8F show inspection of the implant and tissue integration. Black arrows show fibroses, while white arrows show inflammation.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure provides a tissue scaffold implant device for an animal (human or other animals) that serves to expand a lumen or passageway, particularly one that is experiencing stenosis or narrowing. In certain aspects, the tissue scaffold implant device may be used for widening a stenotic lumen in humans or other animal subjects, including mammalian animals, such as livestock, horses, cats, dogs, and the like. As used herein, the term “subject” or “patient” includes humans or animals, unless otherwise specified. Lumens include passageways or physical structures within an organism that in a healthy condition permit fluid to flow therethrough, such as air, carbon dioxide, blood, plasma, and the like. Such a lumen or passageway within a subject in a healthy condition permits fluid flow there through. In certain variations, the lumen may be a tubular tissue structure, for example, one that is selected from the group consisting of: an airway (cricoid, trachea, and bronchi), digestive tract (an esophagus), a blood vessel, lymphatic system, nerves and combinations thereof.

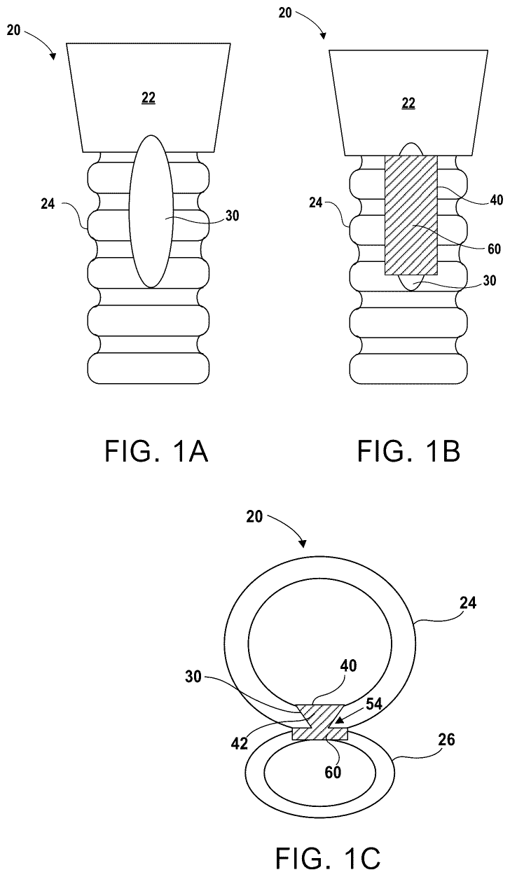

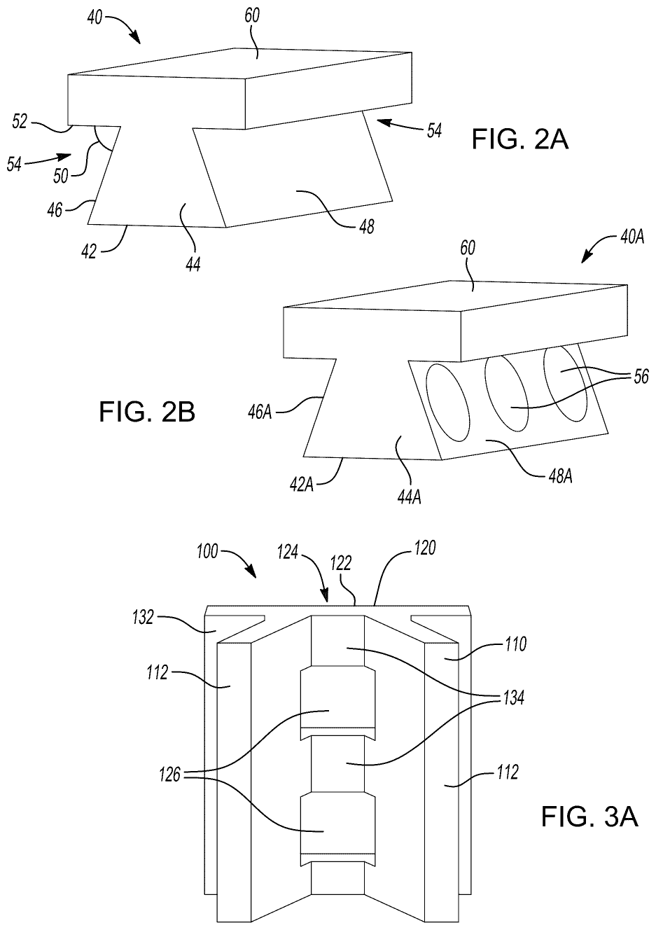

In various embodiments, the tissue scaffold implant device is capable of being placed within an opening formed in the lumen and adjacent to at least a portion of a lumen, for example, adjacent to an external circumference of the lumen, within the subject. By way of example, FIGS. 1A-1C and 2A show an example of a simplified version of a tissue scaffold implant prepared in accordance with certain aspects of the present disclosure that is placed in an opening of a stenotic lumen. For example, a stenotic lumen 20 defines an airway passage including a cricoid 22 and trachea 24. While not shown in FIGS. 1A-1C, at least one region of the stenotic lumen 20 suffers from a narrowing of the passageway inhibiting fluid flow. As shown in a cross-sectional view, the trachea 24 is adjacent to an esophagus 26. During the implantation procedure, first an incision is made in the tissue of the stenotic lumen 20 to form an opening 30. As shown by way of non-limiting example, the opening 30 extends into a portion of the cricoid 22 and a portion of the trachea 24. Then, a tissue scaffold implant device 40 is inserted within the opening 30 in the stenotic lumen 20, so that the tissue scaffold implant device 40 broadens or spreads the tissue to create a wider inner diameter and minimize or reverse the stenosis. This type of tissue scaffold implant may be referred to as a posterior cricoid split tissue scaffold. It will be appreciated that the tissue scaffold implant device 40 may have other orientations to those shown herein. Orientation could be horizontal, allowing for continued cricoid expansion, even after scaffold resorption.

In various aspects, the present disclosure provides a tissue scaffold implant for expanding a stenotic lumen that includes both a bridge structure defining at least two angled sides and a lateral anchor portion integrally formed with the bridge structure. The lateral anchor is configured to be disposed against at least a portion of an external circumference of the stenotic lumen in a region near an opening formed in the stenotic lumen. The tissue scaffold implant defines at least two seat regions are concave regions defined between the at least two angled sides of the bridge structure and the lateral anchor. The at least two seat regions are each configured to be received within and support the opening within the stenotic lumen.

One such variation of such a tissue scaffold implant is shown in FIG. 2A. The tissue scaffold implant device 40 includes a bridge structure 42 defining a solid body 44 that defines at least two angled sides, namely a first angled side 46 and a second angled side 48.

A lateral anchor portion 60 is integrally formed with the bridge structure 42. The lateral anchor portion 60 defines a rectangular shape. The lateral anchor 60 is configured to be disposed against at least a portion of an external circumference of the stenotic lumen 20 in a region near the opening 30, as shown in FIGS. 2B-2C. The lateral anchor portion 60 serves as a retaining vehicle and thus may take any number of forms, including a straight back, as well as partial cylindrical or horseshoe sleeve structures that may be used to secure tissue scaffold implant to the outside of the tubular vessel wall.

For example, in one variation, the tissue scaffold implant device may be used for laryngotracheal reconstruction to expand a narrowed airway by interposing an anterior and or posterior graft into an opening formed in the airway. In another form, the anterior graft tissue scaffold implant device could be a component of an airway splint.

The bridge structure 42 defines a trapezoidal pyramidal shape that is inverted and creates a wedge shape. As shown in FIG. 2A, an angle 50 is defined between each of the first angled side 46 or the second angled side 48 and an inner surface 52 of the lateral anchor portion 60. In certain variations, this angle 50 may be less than or equal to about 120°, for example, greater than or equal to about 20° to less than or equal to about 90°, optionally greater than or equal to about 20° to less than or equal to about 60°, and in one variation, about 56°. In this manner, the tissue scaffold implant 40 defines at least two seat regions 54 that are concave and defined between the at least two angled sides (first angled side 46 or second angled side 48) of the bridge structure 42 and the inner surface 52 of the lateral anchor portion 60. As best seen in FIG. 1C, the concave seat regions 54 receive and support the wall structures of the lumen 20 in the region of the opening 30.

The tissue scaffold implant devices of the present disclosure can support luminal reconstruction with cartilage, perichondrium, bone, muscle, skin, mucosal and dermal grafts, among others to support regrowth and widening of a stenotic lumen. In various embodiments, the tissue scaffold implant device may be formed of a bioacceptable material, which may be a biocompatible or biomedically acceptable metal or polymer that may be biodegradable or non-biodegradable. Such a “biomedically acceptable” material is one that is suitable for use with humans and/or animals without undue adverse side effects (such as toxicity, irritation, and allergic response) commensurate with a reasonable benefit/risk ratio. The term “biodegradable” as used herein means that the implant comprises a polymer that is slowly dissolved or disintegrated under physiological conditions in the human or other animal subject for a certain time and at some point only its degradation products are present in the body in a dissolved or comminuted form. At this point, solid components or fragments of the implant either do not exist anymore or are so small as to be non-harmful or transported away by the subject's circulatory system. The degradation products are substantially harmless in physiological terms and lead to molecules that either occur naturally in the human or other animal subject or can be excreted by the human or other animal subject.

The term “non-biodegradable” bioactive material as used herein means that the biomedically acceptable material will not dissolve in the human or animal subject after implantation. When the materials are polymers, they do not substantially resorb, dissolve or otherwise degrade after implantation in a human or animal subject, under typical physiological conditions. In various embodiments, the tissue scaffold implant of the present technology comprises a polymer, such as a biodegradable polymer. Biodegradable polymers include polycaprolactone, polysebacic acid, poly(octaindiolcitrate), polydioxanone, polygluconate, poly(lactic acid) polyethylene oxide copolymer, modified cellulose, polyhydroxybutyrate, polyamino acids, polyphosphate ester, polyvalerolactone, poly-6-decalactone, polylactonic acid, polyglycolic acid, polylactides, polyglycolides, copolymers of the polylactides and polyglycolides, polye-caprolactone, polyhydroxybutyric acid, polyhydroxybutyrates, polyhydroxyvalerates, polyhydroxybutyrate-co-valerate, poly(1,4-dioxane-2,3one), poly(1,3-dioxane-2-one), poly-para-dioxanone, polyanhydrides, polymaleic acid anhydrides, polyhydroxy methacrylates, fibrin, polycyanoacrylate, polycaprolactone dimethylacrylates, poly-3-maleic acid, polycaprolactone butyl acrylates, multiblock polymers from oligocaprolactonediols and oligodioxanonediols, polyglycerol dodecanedioate (PGD), polyether ester multiblock polymers from PEG and poly(butlylene terephthalates), polypivotolactones, polyglycolic acid trimethyl carbonates, polycaprolactone glycolides, poly(methyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-(carboxyphenoxy) propane], polyhydroxy pentanoic acid, polyanhydrides, polyethylene oxide propylene oxide, and combinations thereof.

In various embodiments, the tissue scaffold implant device comprising the biodegradable polymer allows the passageway defect to be widened to have a larger diameter, heal naturally and then biodegrade or resorb in the subject. Having the tissue scaffold implant device biodegrade eliminates need for a second surgery for removal of the implanted device and furthermore will not inhibit regrowth of the lumen in adults or growth in children.

In various embodiments, tissue scaffold implant device is designed to have a degradation time that coincides with the healing time of the opening in the subject. “Degradation time” refers to the time for tissue scaffold implant device implanted to substantially and fully dissolve, disintegrate, or resorb. Depending upon the subject and the time needed for recuperation and regeneration of the passageway, the degradation time may be about 3 weeks to about 60 months (5 years), or about 2 months to about 40 months (3.33 years), or about 6 months to about 36 months (3 years), or about 12 months to about 24 months (2 years). In certain embodiments, the preferred degradation time for the tissue scaffold implant according to the present technology is about 6 months to about 36 months (3 years). As noted above, in certain embodiments, a preferred biodegradable polymer used to form tissue scaffold implant device comprises polycaprolactone (PCL) or a PCL composite material, such as one that comprises PCL and polyethylene glycol (PEG), which desirably enables such a degradation time of 6 months to about 36 months (3 years) under normal physiological conditions when implanted in an animal subject.

In certain embodiments, tissue scaffold implant device of the present technology optionally comprises a non-biodegradable polymer. Tissue scaffold implant devices may comprise multiple polymers, including one or more biodegradable polymers, one or more non-biodegradable polymers, and any combinations thereof. Suitable biomedically acceptable non-biodegradable polymers include polyaryl ether ketone (PAEK) polymers (such as polyetherketoneketone (PEKK), polyetheretherketone (PEEK), and polyetherketoneetherketoneketone (PEKEKK)), polyolefins (such as ultra-high molecular weight polyethylene, which may be crosslinked, and fluorinated polyolefins such as polytetrafluorethylene (PTFE)), polyethylene glycol (PEG), polyesters, polyimides, polyamides, polyacrylates (such as polymethylmethacrylate (PMMA)), polyketones, polyetherimide, polysulfone, polyurethanes (PU), polysiloxane (e.g., polydimethylsiloxane (PDMS)), and/or polyphenolsulfones.

In certain variations, the bioacceptable material is selected from the group consisting of: polycaprolactone (PCL), polycaprolactone (PCL) and polyethylene glycol (PEG), polylactic acid (PLA), polyurethane (PU), polyglycerol dodecanedioate (PGD), extracellular tissue matrix, polysiloxane, nickel-titanium alloy (nitinol), polyetheretherketone (PEEK), polyetherketoneketone (PEKK), and combinations thereof. In yet other embodiments, a preferred biodegradable polymer that forms the tissue scaffold implant comprises, or consists of, polycaprolactone (PCL) or a PCL composite material, such as one that comprises PCL and polyethylene glycol (PEG).

In certain aspects, the tissue scaffold implant may be porous or non-porous. In certain aspects, a porosity of the tissue scaffold implant may be greater than or equal to 0% to less than or equal to about 70%.

Tissue scaffold implant devices of the present technology can further comprise one or more bioactive materials. Depending on such factors as the bioactive material, the structure of tissue scaffold implant device, and the intended use of tissue scaffold implant device, the bioactive material may be coated on a surface of tissue scaffold implant device, coated or otherwise infused in pores or openings of tissue scaffold implant device, or mixed or compounded within the polymeric material of tissue scaffold implant device. Bioactive materials can include any natural, recombinant or synthetic compound or composition that provides a local or systemic therapeutic benefit. In various embodiments, the bioactive material promotes healing and growth of a collapsed trachea or a collapsed trachea with stenosis. Bioactive materials among those useful herein include cell adhesion factors, isolated tissue materials, growth factors, peptides and other cytokines and hormones, small molecules, pharmaceutical actives, and combinations thereof. In certain aspects, the bioactive material can be applied to a surface of or within the scaffold for anti-microbial, cell adhesion, or vascularization purposes, by way of non-limiting example. Cell adhesion factors include, for example, the RGD (Arg-Gly-Asp) sequence or the IKVAV (Ile-Lys-Val-Ala-Val) sequence. Isolated tissue materials include, for example, whole blood and blood fractions (such as red blood cells, white blood cells, platelet-rich plasma, and platelet-poor plasma), collagen, fibrin, acellularized dermis, isolated cells and cultured cells (such as hemopoietic stem cells, mesenchymal stem cells, endothelial progenitor cells, fibroblasts, reticulacytes, adipose cells, and endothelial cells). Growth factors and cytokines useful herein include transforming growth factor-beta (TGF-β), including the five different subtypes (TGF-β1-5); bone morphogenetic factors (BMPs, such as BMP-2, BMP-2a, BMP-4, BMP-5, BMP-6, BMP-7 and BMP-8); platelet-derived growth factors (PDGFs); insulin-like growth factors (e.g., IGF I and II); fibroblast growth factors (FGFs), vascular endothelial growth factor (VEGF), epidermal growth factor (EGF) and combinations thereof. Examples of pharmaceutical actives include antimicrobials, antifungals, chemotherapeutic agents, and anti-inflammatoires. Examples of antimicrobials include triclosan, sulfonamides, furans, macrolides, quinolones, tetracyclines, vancomycin, cephalosporins, rifampins, aminoglycosides (such as tobramycin and gentamicin), and mixtures thereof. In certain variations, an implantable splinting device comprises acellularized dermis, an acellularized tissue matrix, a composite of acellularized dermis matrix and designed polymer, and/or a composite of acellularized tissue matrix and designed polymer. In certain aspects, an implantable splinting device comprises an acellularized dermis layer disposed on one or more surfaces that will contact the passageway upon implantation.

In certain variations, a bioactive material may be selected from the group consisting of: an isolated tissue material, a hydrogel, an acellularized tissue matrix, a composite of acellularized tissue matrix and designed polymer, and combinations thereof. An isolated tissue material may include an autologous or allogeneic tissue sample (such as an explant harvested in the patient by a punch biopsy to provide tissue sample). In other aspects, an isolated tissue material may include isolated or cultured cells (such as chondrocyte cells, hemopoietic stem cells, mesenchymal stem cells, such as adipose-derived mesenchymal stem cells, endothelial progenitor cells, fibroblasts, reticulacytes, and endothelial cells), whole blood and blood fractions (such as red blood cells, white blood cells, platelet-rich plasma (PRP), and platelet-poor plasma, bone marrow aspirate), collagen, fibrin, acellularized tissue, and the like. In certain variations, the tissue or bioactive material inserted into the tissue scaffold implant device may include, but is not limited to, cartilage, perichrondrium, fibrous tissue, muscle, adipose, adipose derived stem cells, mesenchymal stem cells, bone marrow aspirate, blood or plasma/platelet-rich plasma (PRP) and other tissues to provide a cell sources for healing. In one embodiment, the isolated tissue biomaterial may comprise perichondrium, cartilage, adipose, or combinations thereof. Cartilage may be obtained from ear, thyroid, or most commonly rib autologous donor sites. In addition, autogenous and autologous cells may also be delivered from the main body of the tissue scaffold implant device. Finally, proteins and/or genes may be functionalized onto one or more surfaces of the tissue scaffold implant devices.

Hydrogels are materials formed from lightly-crosslinked networks of natural or synthetic polymers, such as saccharides, which have high water contents such as 90% weight per volume or greater. Hydrogel crosslinking can be achieved by various methods including ionic, covalent chemical, or UV-initiated chemical crosslinking. Hydrogels used in the present disclosure are preferably biocompatible. Hydrogels may be formed from hyaluronic acid/hyaluronan, sodium alginate, polyethylene glycol (PEG), polyethylene glycol diacrylate (PEGDA), 2-hydroxyethyl methacrylate (HEMA)/poly(2-hydroxyethyl methacrylate) (pHEMA), polymethyl methacrylate (PMMA), polyacrylic acid, chitosan, poly(amino acids), poly(N-isopropylacrylamide) (PNIPAM), collagen, gelatin, fibronectin, chondroitin sulfate, surfactant gels (having greater than about 20% weight per volume poloxamers (e.g., commercially available as PLURONIC™ and BRIJ™), polydimethylsiloxane (PDMS) or dimethicone, epoxy, polyurethane, and the like. In certain variations, a hydrogel can be incorporated onto or into the tissue scaffold implant to deliver other bioactive agent molecules, like small molecules or other biologics for anti-microbial, cell adhesion, or vascularization purposes, by way of non-limiting example.

FIG. 2B shows an alternative version of a tissue scaffold implant 40A. To the extent that the features are the same as those shown in FIG. 2A, they share the same reference numbers and will not be discussed further herein unless otherwise warranted. A bridge structure 42A includes a body 44A that defines at least two angled sides, namely a first angled side 46A and a second angled side 48A. Like the bridge structure 42 in FIG. 2A, the bridge structure 42A has a trapezoidal pyramidal shape. Further, the body 44A defines a plurality of openings or apertures 56 that extend from the first angled side 46A to the second angled side 48A. The openings 56 can provide regions to permit ingrowth of surrounding tissue from the lumen 20. Alternatively, the openings 56 may be filled with a tissue graft or a bioactive material (not shown, but as described above). By way of non-limiting example, the openings 56 may receive cartilage punch biopsy tissue inserts.

For example, one or more tissue samples may be harvested from the patient (or from another source of tissue) via a punch biopsy tool or other technique. The punch biopsy tool may be dimensioned to provide tissue samples that will seat within the hollow feature or opening 56. In certain variations, the tissue harvested is cartilage or perichondrium tissue. After harvesting, the tissue sample may be disposed within the openings, like openings 56, of the scaffold implant. Thus, the tissue scaffold implant may be seeded by implanting tissue (e.g., cartilage) punches prior to implantation into the patient. Custom punch biopsy designs and accompanying scaffold inserts allow for rapid and precise harvest and distribution of tissue within the scaffold when the tissue sample is disposed in the one or more hollow features or openings. The scaffold design allows for precise placement and distribution of cartilage punches. In certain variations, a plurality of hollow features or openings may be provided in various regions of the bridge structure that supports and contacts the adjacent lumen tissue walls.

The size and distribution of hollow features or openings for accepting tissue (e.g., cartilage) punch biopsy inserts can be determined based on Finite Element Analysis to guide relief of overlying soft tissue strain and vascular compromise. In certain variations, the tissue scaffold implant may have the capability of having varying diameters in stacked or multilayer conformation. In certain variations, the opening that accepts prefabricated inserts may have a diameter of greater than or equal to about 500 micrometers (μm) to less than or equal to about 15 mm, optionally greater than or equal to about 1 mm to less than or equal to about 10 mm.

The tissue scaffold implant device body may contain large pores or holes to incorporate tissue biopsies. Porous tissue implant scaffold designs radiating from prefabricated openings that accept punch biopsy inserts facilitate cellular, paracrine, and autogenous growth factor dissemination. Further, an eluting periinsert component allows for gradual dissolution of the tissue sample/cartilage punch, further facilitating cellular outgrowth, paracrine influence. Thus, a macropore hollow feature or opening configured to accept tissue biopsy punches, like cartilage or perichondrium, advantageously promotes cellularization of the tissue scaffold, while minimizing operative time and accelerating translation. Punch biopsy insert sites in the tissue scaffold implant may additionally have function altering the tissue insert, for example, optimizing tissue distribution within the scaffold, such as having a serrated edge to splay and distribute the tissue.

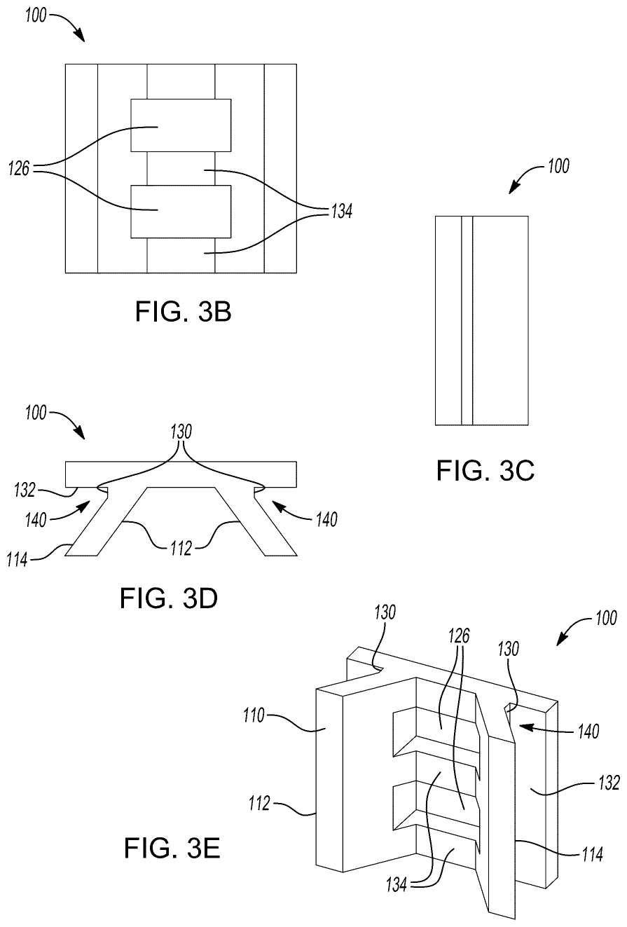

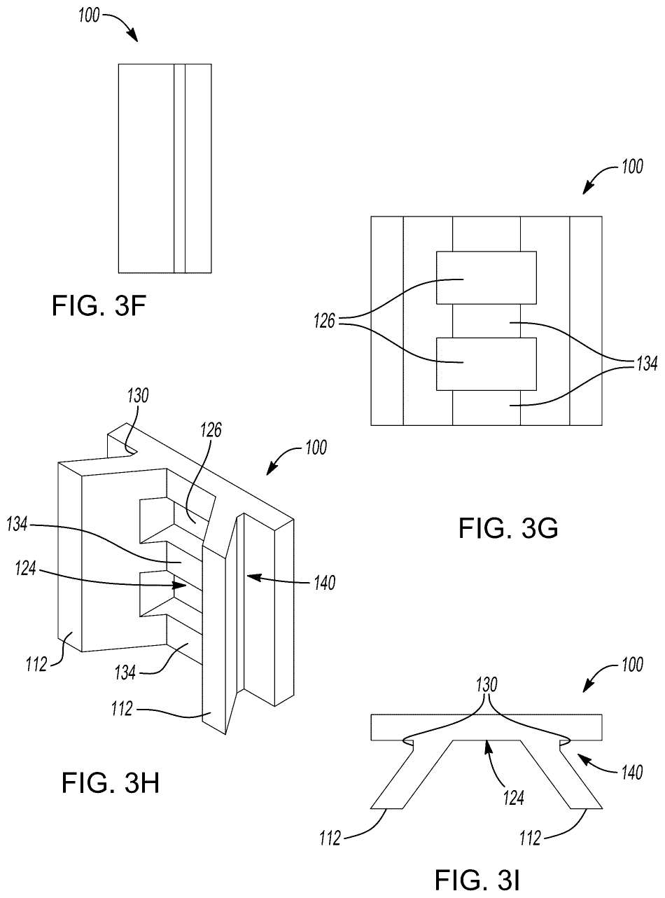

FIGS. 3A-3I show another variation of a tissue scaffold implant 100 having a lighter weight fillet and truss design. As best seen in FIGS. 3D-3E and 3H-3I, the tissue scaffold implant 100 defines a “K-shape.” The tissue scaffold implant device 100 includes a bridge structure 110. The bridge structure 110 comprises two angled walls 112 that define wings. The angled walls 112 thus define at least two angled sides 114.

A lateral anchor portion 120 is integrally formed with the bridge structure 110. The lateral anchor 120 defines a rectangular shape. The lateral anchor 120 is configured to be disposed against at least a portion of an external circumference of a stenotic lumen in a region near the opening formed in the lumen. In this variation, the lateral anchor 120 defines a solid body 122 having a central region 124. The central region 124 includes at least one hollow region, namely, a plurality of openings 126 having a rectangular shape. In this manner, the lateral anchor 120 defines a trussed structure that is lightweight, while providing lateral structural support to the tissue scaffold implant 100. The trussed structure of the lateral anchor 120 thus defines a plurality of trusses or struts 134 between the plurality of openings 126 in the central region 124. In certain aspects, the tissue scaffold implant 100 can exert a balanced radial force in an axial plane restoring native lumen size of a passageway. For example, the tissue scaffold implant may exhibit enough mechanical stiffness and strength to expand the posterior glottic aperture. In one non-limiting example, the tissue scaffold implant 100 may have a strength (e.g., ultimate tensile strength) of greater than or equal to about 0.1 MPa to less than or equal to about 10 MPa.

Furthermore, a fillet or concave junction 130 is respectively defined between an inner surface 132 of the lateral anchor structure 120 and each of the two angled sides 114 of the two wings/two angled walls 112 to define the at least two seat regions 140 configured to receive walls of a stenotic lumen near the opening formed therein. While non-limiting, the concave junction 130 defines an angle between the angled side 114 and the inner surface 132 of about 56°, notably forming a corresponding angle in the inner central region 124 of about 126°.

In the variation shown in FIGS. 3A-3I, examples of suitable dimensions are shown, which of course may vary based on the patient in which the implant is placed as recognized by those of skill in the art, for example, a pediatric versus adult patient, the diameter and dimensions of the stenotic lumen, and the like. In this non-limiting example, as best seen in FIGS. 3A and 3F-3G, an 3I, a height of the tissue scaffold implant 100 may be greater than or equal to about 2 mm to less than or equal to about 60 mm, optionally about 10 mm in the embodiment shown, while a width (measured along the entire back of the lateral anchor 120) may be greater than or equal to about 1 mm to less than or equal to about 25 mm, optionally about 11 mm in the embodiment shown. A width of the central region 124 extending to each concave junction 130 is about 7 mm, while terminal ends are each about 2 mm wide. An overall height or thickness of the tissue scaffold implant 100 (anterior to posterior), including the lateral anchor 120 and the bridge structure 110 may be greater than or equal to about 1 mm to less than or equal to about 20 mm, optionally about 4.5 mm in the embodiment shown. Each angled wall 112 has a width of about 1.5 mm and a length of about 4 mm.

In certain embodiments, tissue scaffold implant devices prepared in accordance with the present disclosure may have a length ranging from greater than or equal to about 2 mm to less than or equal to about 60 mm, optionally greater than or equal to about 6 mm to less than or equal to about 60 mm. When the tissue scaffold implant device is used for a pediatric patient, the length may range from greater than or equal to about 2 mm to less than or equal to about 30 mm.

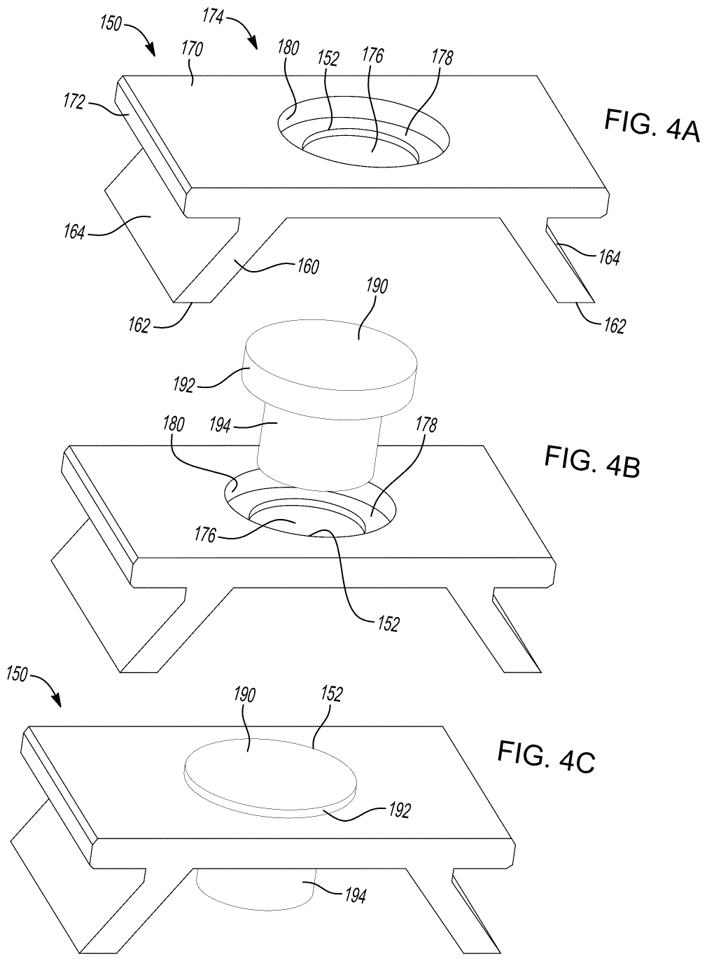

FIGS. 4A-4C show another variation of a tissue scaffold implant 150 having a cross-sectional “K-shape” with a holder 152 for receiving a cylindrical biopsy that can include both perichondrium (top) and cartilage (bottom) tissue. The tissue scaffold implant device 150 includes a bridge structure 160. The bridge structure 160 comprises two angled walls 162 that define wings. The angled walls 162 thus define at least two angled sides 164. The at least two angled sides 164 further define a textured or patterned surface that may enhance friction forces and retention of the walls of the stenotic lumen. The texture or pattern may be serrations, toothed patterns, or corrugations, by way of example. As will be appreciated, any of the designs described herein having a surface along an angled side may be textured or patterned to enhance retention of the walls of the opened stenotic lumen.

A lateral anchor portion 170 is integrally formed with the bridge structure 160. The lateral anchor 170 defines a solid body having a rectangular shape. The lateral anchor 170 is configured to be disposed against at least a portion of an external circumference of a stenotic lumen in a region near the opening formed in the lumen. In this variation, the lateral anchor 170 defines a solid body 172 having a central region 174. The central region 174 includes a hollow region or opening 176 that defines the holder 152. The opening 176 has a circular shape and defines a first diameter. The holder 152 includes a circumferential flange 178 that defines the first diameter. As best shown in FIGS. 4B-4C, the opening 176 also defines an upper region 180 having a second diameter that is a larger diameter than the first diameter. In this manner, the circumferential flange 178 serves as a seat for a tissue insert 190 that includes both an upper region 192 with perichondrium tissue and a lower region 194 with cartilage tissue. Such a tissue insert 190 can be obtained by taking a punch biopsy into a patient, where the harvesting biopsy device has different diameters. Thus, the upper region 192 has a diameter that is less than the second diameter of the upper region 180 of holder 152, but greater than the first diameter of the circumferential flange 178, so that it will seat in the holder 152. In certain variations, the first diameter of the circumferential flange may be greater than or equal to about 1 mm to less than or equal to about 10 mm, while the second diameter of the upper region 180 of the holder 152 may be greater than or equal to about 2 mm to less than or equal to about 20 mm. The lower region 194 of the tissue insert 190 has a diameter than is less than both the first diameter of the circumferential flange 178 and the second diameter of the upper region 180. As best shown in FIG. 4C, the tissue insert 190 can be pushed into opening 176 and seated in the holder 152.

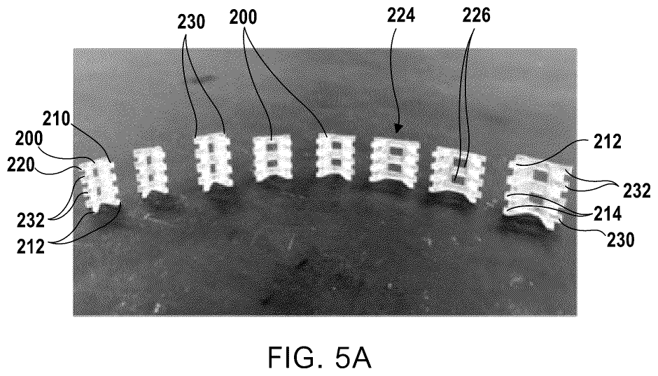

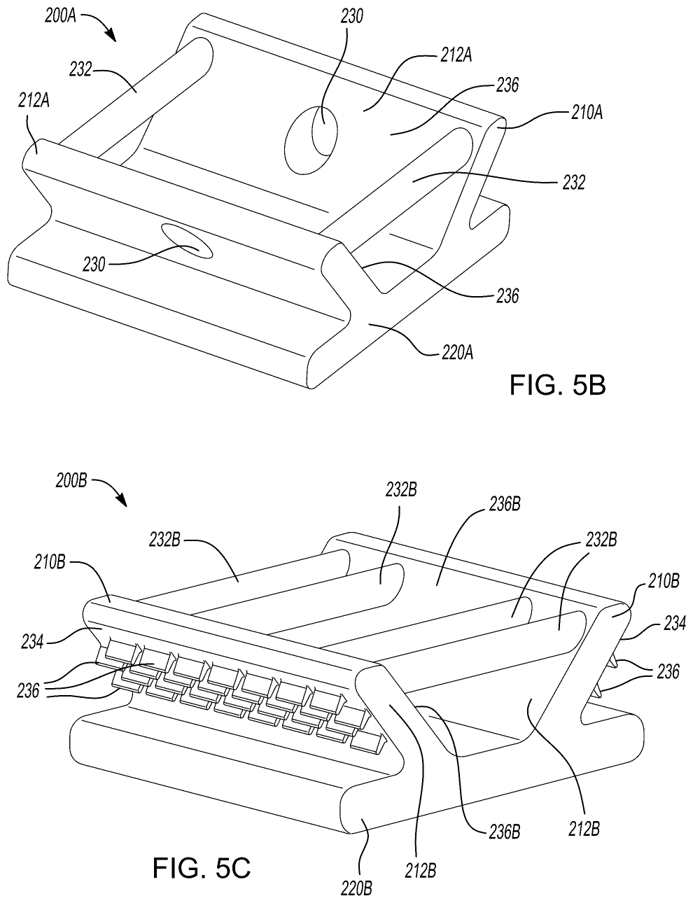

FIG. 5A shows yet other variations of tissue scaffold implants 200 having a variety of different widths and heights, while also embodying a lighter weight design. Like the embodiment in FIGS. 3A-3I and 4A-4C, the tissue scaffold implants 200 likewise define a cross-sectional “K-shape.” The tissue scaffold implants 200 include a bridge structure 210 and a lateral anchor 220. The bridge structure 210 comprises two angled sides 212. The two angled sides 212 each comprise a plurality of angled support teeth 214 spaced apart from one another by a predetermined distance, forming an angled comb-like structure.

A lateral anchor portion 220 is integrally formed with the bridge structure 210. The lateral anchor 220 generally defines a rectangular shape. The lateral anchor 220 is configured to be disposed against at least a portion of an external circumference of a stenotic lumen in a region near the opening formed in the lumen. In this variation, the lateral anchor 220 defines a structure having a central region 224. The central region 224 includes at least one hollow region, namely, a plurality of openings 226 having a rectangular shape. Further, the lateral edges 230 of the lateral anchor 220 comprise a plurality of teeth 232 spaced apart from one another by a predetermined distance, forming a comb-like structure along each lateral edge 230. In this manner, the lateral anchor 220 defines a trussed structure that is lightweight, while providing lateral structural support to the tissue scaffold implant 200. The trussed structure of the lateral anchor 220 thus defines a plurality of struts in the central region 224 that further create a lattice framework extending between the lateral edges 230. In this tissue scaffold device design, there is a reduced amount of material present that can allow more rapid mucosalization and healing. Further, the trussed structure may also add security to placement or better anchoring of the tissue scaffold implant.

As shown in FIG. 5B, another variation of a tissue scaffold implant 200A is shown. The tissue scaffold implant 200A defines the cross-sectional “K-shape,” but further has longitudinal support structures to reinforce the K-shape to prevent inward collapse, as described herein. A bridge structure 210A comprises two angled sides 212A. The two angled sides 212A each comprise at least one opening or aperture 230 in each solid wall defining the two angled sides 212A. In this manner, the two angled sides 212A provide structural support to the tissue scaffold implant 200A while reducing weight.

Further, the bridge structure 210A has a plurality of support structures 232 that span from one angled side 212A to the other side. Such support structures 232 may be structs and/or fillets that can be included in the K-shaped tissue scaffold implant 200A to prevent inward collapse of the graft. As such, two support structures 232 are disposed in a superior position and an inferior position, although the support structures 232 are not limited to this number or particular placement when bridging the walls defined by the angled sides 212A.

Like other designs, a lateral anchor portion 220A is a solid base that is integrally formed with the bridge structure 210A. The lateral anchor 220A generally defines a rectangular shape in this variation and is configured to be disposed against at least a portion of an external circumference of a stenotic lumen in a region near the opening formed in the lumen.

FIG. 5C shows yet another variation of a tissue scaffold implant 200B. The tissue scaffold implant 200B defines the cross-sectional “K-shape,” but further has longitudinal support structures to reinforce the K-shape to prevent inward collapse, as described herein. Further, the tissue scaffold implant 200B has a plurality of securement features, which are shown as angled support teeth. A bridge structure 210B comprises two angled sides 212B. The two angled sides 212B each define a solid wall.

The bridge structure 210B has a plurality of support structures 232B that span from one angled side 212B to the other side. Like the support structures 232 in FIG. 5B, such support structures 232B may be longitudinal supports, such as struts and/or fillets, that can be included in the K-shaped tissue scaffold implant 200B to prevent inward collapse of the graft. In this variation, two pairs of support structures 232B (four support structures 232B) are disposed in a superior position and an inferior position, although again the support structures 232B are not limited to this number or particular placement when bridging the walls defined by the angled sides 212B.

Each angled side 212B defines an outward facing surface 234 that may be a lateral cricoid facing plane. As this tissue scaffold implant 200B design in FIG. 5C typically does not permit a clinician/surgeon to suture in place, the securement features allow improvement in security once placed during the implantation procedure. However, such securement features may also be used in tissue scaffold implants that also have the ability to be sutured, providing an additional measure of securement after placement. Of note, the preliminary sizing grafts may not include these features, but a final graft for implantation could include these securement features.

Thus, each angled side 212B in this variation comprises a plurality of securement features in the form of angled support teeth 236 spaced apart from one another by a predetermined distance for grasping tissue, forming an angled comb-like structure. Directionality of the angled support teeth 236 features allow for graft insertion, but resistance to extrusion, and thus are posterior or lateral facing. Alternative securement features on the outward facing surface 234 (e.g., lateral cricoid facing planes) may include projections, textures, patterns, high surface roughness, or scale features to promote grasping or gripping of and securing to adjacent tissue. As discussed above, a textured, patterned, or rough surface may enhance friction forces and retention of the walls of the stenotic lumen. The texture or pattern may be serrations, toothed patterns, or corrugations, by way of example These features may be designed as a component of the manufactured tissue scaffold or alternatively may be produced by post processing methods, such as laser etching, plasma treatment and/or chemical treatment to modify the surface.

Like other designs, a lateral anchor portion 220B is a solid base that is integrally formed with the bridge structure 210B. The lateral anchor 220B generally defines a rectangular shape in this variation and is configured to be disposed against at least a portion of an external circumference of a stenotic lumen in a region near the opening formed in the lumen.

While not limited to these embodiments, but merely for purposes of illustration, with reference to the tissue scaffold implant 200A and tissue scaffold implant 200B in FIGS. 5B and 5C, each angled side 212A or angled side 212B defines an inward facing surface 236 on angled sides 212A of bridge structure 210A in FIG. 5B and an inward facing surface 236B on angled sides 212B of bridge structure 210B in FIG. 5C. The inward facing surfaces 236 and 236B may each be an anterior airway facing surface that may be further modified to enhance growth and incorporation of the tissue implant after implantation. For example, the inward facing surfaces 236 and 236B may be modified by having a composite overlay of hydrogel, chemical treatment, and/or biologic treatment. Surface porosity or design on the surfaces may also be optimized to facilitate rapid mucosalization, vascularization, and healing. Further, surface modifications may also impart antimicrobial properties to minimize risk of infection, for example, by applying an antimicrobial material as a coating or incorporating an antimicrobial material into predetermined portions of the body of the implant.

With renewed reference to FIG. 5A, a variety of tissue scaffold implants 200 having different heights and widths that can be formed for use in a variety of applications depending on the nature of the stenotic lumen and its dimensions. The tissue scaffold implants 200 may be customized to a specific patient by using an image-based design approach specific to a human or animal subject or may be selected from various standard sizes (widths, lengths, and heights) for a specific patient or subject, for example, a pediatric patient or an adult. In certain aspects, the present disclosure may contemplate a kit for a surgical procedure to widen a stenotic lumen.

Inclusion of predefined punch biopsy inserts allows for relatively inexpensive and easy to use airway tissue engineering. The kits may include existing instruments, such as dermal punch biopsies, so that surgeons would then have the capability to impart cellularization of the tissue scaffold with ease.

The kit may include a plurality of distinctly sized tissue scaffold implants 200 from which the surgeon or user of the kit may choose. As shown in FIG. 5A, dimensions of the tissue scaffold implants 200 may vary in both height, length, and width. The dimensions of the tissue scaffold implant 200 can be designed from commonly used widths and lengths specific to a human or animal subject.

In certain variations, the kit may further include a harvesting tool for having a cutting mechanism capable of harvesting a tissue graft from the patient. The cutter is used for cutting and removing a tissue graft from a source of tissue in the subject to form a cylindrical tissue graft. As shown above in FIGS. 4A-4C, this may be a specialized tool that creates a cylindrical tissue graft having two distinct diameters. Cutter components may be interchangeable, disposable, and/or replaceable. Where the cutter component is interchangeable, it may be selected from for a variety of distinctly sized cutter components. Thus, the kit may include a plurality of distinctly sized cutter components (e.g., having a cutting tube with distinct diameters or volumes) from which the surgeon or user of the kit may choose. The cutting device or tool may be omitted (e.g., if previously purchased) from the kit.

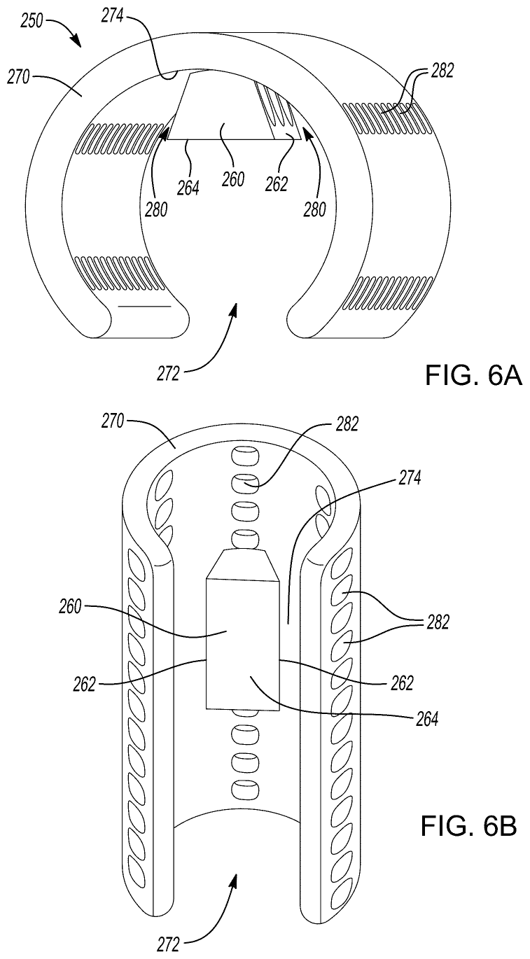

FIGS. 6A-6B show yet another variation of a tissue scaffold implant 250. The tissue scaffold implant device 250 includes a bridge structure 260, similar to that shown in FIGS. 2A-2B, which may share similar features not necessarily discussed herein for brevity. The bridge structure 260 comprises two angled walls that define angled sides 262. The bridge structure 260 defines a trapezoidal pyramidal shape that is inverted and creates a wedge shape to interface with the walls of the stenotic lumen with the opening. The bridge structure 260 also defines an exposed surface 264, which in the case of the tissue scaffold implant device 250 being used as a tracheal splint, may be a posterior, airway facing surface.

A lateral anchor portion 270 is integrally formed with the bridge structure 260. The lateral anchor portion 270 defines a curved sleeve (e.g., a partial cylinder with a longitudinal slit or opening 272) that can be placed around and conforms to at least a portion of an exterior circumference of the stenotic lumen. The lateral anchor 270 is thus configured to be disposed against and to support at least a portion of an exterior (external circumference) of the stenotic lumen in a region near the opening. In this manner, the lateral anchor portion 270 may support a passageway/lumen that is weakened or has a potential risk of collapse.

The tissue scaffold implant 250 defines at least two seat regions 280 defined between the at least two angled sides 262 of the bridge structure 260 and an inner surface 274 of the lateral anchor portion 270. The seat regions 280 receive and support the wall structures of the lumen in the region of the opening. The lateral anchor portion 270 has a plurality of openings 282 formed therein, which can serve as holes for receiving needles to create sutures to affix the sleeve to the stenotic lumen during surgery. The openings 282 can be pre-aligned to fit a surgical needle to allow for suturing to the affected lumen during surgery. The openings 282 configured to accept the suture needle may have a diameter ranging from greater than or equal to about 0.5 mm to less than or equal to about 3 mm.

Once the tissue scaffold implant device is placed around the passageway, such as the trachea, the bronchi, the esophagus or the blood vessel of the patient, the implant device is sutured to the area using a portion of a plurality of openings configured to accept the suture needle. Notably, it is contemplated that implantable splinting devices according to certain aspects of the present teachings may have different numbers of apertures or openings for suturing to the stenotic lumen during implantation and the number and placement of the openings/apertures is non-limiting. As with any of the embodiments described above or herein, in certain variations, the tissue scaffold implant may comprise one or more apertures for receiving a suture or temporary safety stitch. For example, in one variation, a predefined superiorly placed surgical needle hole allows for placement of a safety stitch that is cut after ensuring the tissue graft implant is securely placed. Such an aperture may have an average diameter as specified above, for example, greater than or equal to about 0.5 mm to less than or equal to about 3 mm.

Further, as with any of the embodiments described above or herein, in certain variations, the tissue scaffold implant may have one or more modified surfaces to promote tissue growth, reduce infection, and the like as described above in the context of the inward facing surfaces 236, 236B (e.g., anterior airway facing surfaces) in FIGS. 5B and 5C. By way of example, in an anterior airway graft design, lateral tracheal facing surfaces similarly may be optimized as described above to facilitate mucosalization, vascularization, healing, and/or to promote antimicrobial properties.

Modifications may also be included to carry and deliver methods for creating air and water tight seals, such as providing fibrin glue sealant, in more precise delivery systems than currently used (e.g., angiocath spray over top of the reconstruction). The lateral facing surfaces may further have a composite material (either as a surface layer or being formed from a composite material) in the lateral facing surfaces to achieve fluid-tight (e.g., air and water tight) seals. For the anterior airway graft used as a component of a tracheal splint in FIGS. 6A-6B, the exposed surface 264 of bridge structure 260 may be a posterior, airway facing surface posterior, airway facing surface that may also have the aforementioned optimizations to facilitate rapid mucosalization, vascularization, prevent infection, and optimize healing. Lateral surface modifications may additionally allow enhanced securement and positioning in the anterior position. In certain further variations, an overhang may be used to aid in maintenance of position of the recipient airway wall. This may ultimately allow placement of anterior airway grafts without the need for suture securement. Alternatively, prepositioned sutures may be incorporated into the tissue scaffold implant device to allow ease in placement of anterior and posterior airway grafts with a parachute technique.

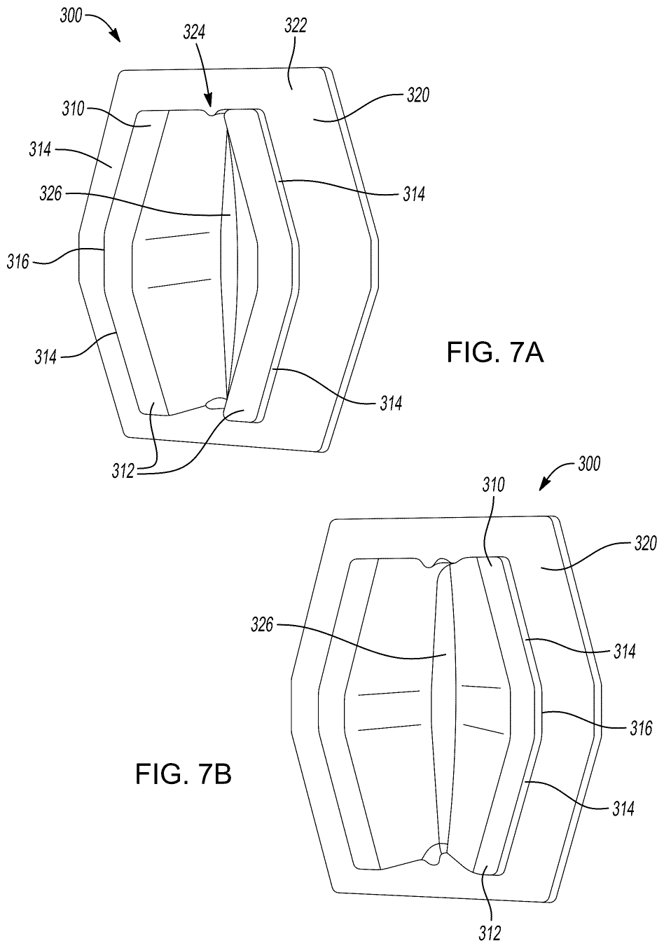

In FIGS. 7A-7B, a tissue scaffold implant 300 has an angled polygonal shape in a form of an elliptic or truncated diamond so that is complementary to a shape of surgically formed opening in a stenotic lumen. The tissue scaffold implant device 300 includes a bridge structure 310 and a lateral anchor portion 320. The lateral anchor portion 320 is integrally formed with the bridge structure 310. In this variation, the lateral anchor 320 defines a solid body 322 having a central region 324. The central region 324 includes a hollow region or opening 326 having a longitudinal rectangular shape. The lateral anchor 320 defines a polygonal shape in the form of a diamond shape having truncated points or ends.

The bridge structure 310 comprises two distinct walls that define wings 312 that each respectively form two different angled sides 314. The bridge structure 310 thus defines at least two angled sides 314 on each wing or wall that are complementary with the polygonal shape (having two diagonal lines joined at an intersection or apex 316).

The use of three dimensional (3D) printing or additive manufacturing allows for rapid prototyping, the use of a wide variety of materials with unique properties, and allows for patient specific design through the use of computer aided design (CAD). Previous studies have also shown that polycaprolactone (PCL), a slowly degrading, printable, bioresorbable material, is safe and efficacious for the use in airway devices and has ideal properties for this application.

An image-based design approach can be used to form a customized tissue scaffold implant by using medical images or other data that is specific to the patient to customize the size of tissue scaffold implant device. The specific medical images and/or parameters are obtained from one or more imaging systems such as computed tomography (CT), a CT-fluoroscopy, fluoroscopy, magnetic resonance imaging (MRI), ultrasound, positron emission tomography (PET) and X-Ray systems or any other suitable imaging systems. The medical image data and/or parameters received from the imaging system provide a two-dimensional (2D), three-dimensional (3D) or four-dimensional (4D) model of an anatomical structure, organ, system or region of the patient. The image-based design of the 2D, 3D or 4D model may be created using MATLAB®, Mathematica®, or other CAD software design programs known in the art. For converting the design into a usable format for rapid prototyping and computer-aided manufacturing, a STL file format may be created. This file format is supported by many software packages such as Mimics® by Materialise, MATLAB®, IDL, and Amira®.

This 2D, 3D or 4D model of tissue scaffold implant device may then be used to manufacture a tissue scaffold implant device. Methods of manufacturing can include a variety of 3D printing/additive manufacturing methods (laser sintering, fused deposition modeling, nozzle based systems). The device made by a variety of suitable methods, including methods comprising solid free-form fabrication (SFF) techniques such as laser sintering, stereolithography, 3D printing and injection molding. In various embodiments, the preferred method is laser sintering. Laser sintering is a process involving the construction of a three-dimensional article by selectively projecting a laser beam having the desired energy onto a layer of particles. The laser sintering process can be paired with medical image data and/or parameters received from the imaging system for producing a customized implantable splinting device of the present technology.

Using an image-based approach, all dimensional aspects of the graft/tissue scaffold implant can be specified including the support bridge dimensions (e.g., trapezoidal base and top height and width), the lateral anchor base height and width, as well as the length of overall graft/tissue scaffold implant. Furthermore, the dimensions of all the components may be functionally graded by the user to create shapes to accommodate the incision shape

In various aspects, the present technology contemplates using tissue scaffold implant devices like those described above, where the bridge support structure may be inserted as a wedge via a surgical implantation procedure into a stenotic tubular tissue to broaden that tissue. An incision is made in the stenotic lumen tissue and the implant device is inserted to broaden or spread the tissue due to the presence of the bridge support. The lateral anchor serves as a retaining vehicle for the implant, which may take a number of forms, including a straight back as well as partial cylindrical or horseshoe structures that may be used to secure the main implant body to the outside of the tubular vessel wall.

Various embodiments of the inventive technology can be further understood by the specific examples contained herein. Specific Examples are provided for illustrative purposes of how to make and use the devices, compositions, and methods according to the present teachings.

### EXAMPLES

### Example 1

The present example forms a novel bioresorbable scaffold based on optimized design features of the costal cartilage grafts used in conventional procedures. The design targets for the new scaffold are as follows; 1) the new scaffold could eliminate the need for rib cartilage harvesting; 2) the scaffold can maximize the airway cross-sectional area once implanted; 3) the scaffold design may allow for scaling to different sizes; 4) the scaffold can provide enough mechanical stiffness and strength to expand the posterior glottic aperture; 5) the scaffold will be made of a biocompatible material that will not require future procedural removal; and 6) the scaffold is designed to minimize implanted material to encourage rapid mucosalization.

### Tissue Scaffold Implant Design

Initial scaffold dimensions were taken from a mean of measurements of a costal graft carved in simulated rib cartilage by an experienced Otolaryngology Head and Neck Surgeon. These measurements were used to create a computer aided design (CAD) file on Materialise 3Matic Medical software V11.0. To meet design criteria (2) above, the luminal face of the scaffold was imparted concavity, allowing a larger airway lumen. The backbone of the scaffold was then modified to give a range of sizes which allowing for a range of dimensions to accommodate patient ages and body habitus and also to allow surgeons to have multiple options immediately available for them to select from. See FIG. 5 above. This will also allow for the use of a “dummy graft” that would allow real time sizing prior to implant selection. The CAD models were then exported as .STL file, which can be used to guide any three-dimensional (3D) printer.

The scaffolds were manufactured from polycaprolactone (PCL) with 4% hydroxyapatite (HA) using laser sintering (Formiga P 110 system; EOS e-Manufacturing Solutions), a 3D printing technology that has been adapted to use PCL to build complex medical implants and anatomic 3D structures. Approximately 200 posterior cricoid scaffolds may be built in 4 hours using this manufacturing technology. The 3D printed PCL scaffolds were selected from the series of different sizes during the procedure by the lead surgeon, as will be described further herein. The scaffolds maintained their structural integrity and successfully expanded the cricoid cartilage and glottic inlet in all the animal subjects during implantation as verified by post-closure endoscopy.

The implementation of the 3D printed PCL scaffolds eliminated the need for costal cartilage harvesting in all three animals in the completion of this procedure. Surgical times from first incision to complete closure were 110 min, 95 min, 100 min in each of the respective procedures. These surgical times allowed for comprehensive photographic and video documentation and a comfortable surgical pace with interspersed medical student education.

### Surgical Models

As a pilot proof of concept large animal model study, three Yorkshire pigs were implanted with posterior cricoid scaffolds and followed for six weeks post-implantation. Institutional Animal Care and Use Committee (IACUC) approval was obtained through T3 laboratories. The Yorkshire pig was used as a preclinical animal model because the porcine trachea has biomechanical and anatomic properties similar to those of the growing human airway.

Pre-procedure, intramuscular tiletamine 4 mg/kg, xylazine 0.5 mg/kg, carprofen 3 mg/kg, buprenorphine 0.01 mg/kg and intravenous cefazolin 22 mg/kg was given for sedation, analgesia and antibiotic prophylaxis. 0% to 5% inhaled isoflurane was used to maintain anesthesia throughout the procedure. Body temperature, heart rate, and breathing rate as well the palpebral reflex were monitored to assure the appropriate depth of anesthesia. An open approach was utilized after initial attempts at endoscopic approach were felt to be logistically difficult due to porcine airway distance from the operator. The cervical skin was prepared and draped in sterile fashion. An anterior cervical approach via a vertical, midline skin incision over the larynx and trachea was performed. The sternothyroid and sternohyoid musculature were dissected and retracted laterally, providing wide exposure to the laryngotrachea. A temporary tracheostomy was placed to allow the previously placed endotracheal tube to be withdrawn. The first and second tracheal rings along with the anterior tracheal mucosa were dissected and retracted laterally to allow for visualization of the posterior cricoid and trachea.

The posterior cricoid cartilage was defined and incised. The PCL scaffolds were placed and secured with 5-0 PDS suture using parachuting technique. The trachea was closed, the subject was reintubated, the tracheotomy was closed, and the surgical incision was closed loosely in layered fashion with a Penrose drain in place. Post-procedure the pigs were given oral cephalexin 22-25 mg/kg twice a day for seven days. Intramuscular carprofen 3 mg/kg once daily for five days and buprenorphine 0.01 mg/kg twice daily for three days was given for analgesia.

### Clinical Outcome Assessments

Postoperatively, temperature, appetite, behavior, and tenderness at the incision and implant sites were monitored. Furthermore, the validated Westley Croup Scale was used for daily clinical assessments of the animals. Any decision for early termination was made based on clinical evaluation by a laboratory veterinarian. Endoscopic evaluation was performed to grossly inspect the trachea prior to termination when possible. Necropsy was performed to determine cause of death if applicable and included harvesting of the trachea along with implanted scaffolds for histological evaluation.

The whole larynx was removed from pigs at endpoints and an incision was carefully made in the anterior thyroid cartilage. The laryngeal tissues were separated to expose the implantation site, which was examined visually for inflammation and infection. Tissue samples containing the implanted posterior cricoid were trimmed and visually inspected for damage, degradation and displacement. Samples with intact specimens were immersed in 10% Formalin, paraffin embedded and sectioned. Sectioned tissues were stained with hematoxylin and eosin to evaluate inflammation. Additional adjacent sections were stained with safranin O to evaluate cartilage growth into the implant. A total of n=3 slides were prepared per sample for each stain with 3 sections per slide.

Gross imaging of pig tissues was conducted using a Nikon d60 DSLR camera with a NIKKOR 35-75 mm lens. Histology images were taken on a Zeiss Axioobserver microscope. A total of 4 fields of view with a 20× objective were used to evaluate inflammation in each H+E stained section (n=12/sample). Biocompatibility of the implants was evaluated by scoring histological sections for inflammation, giant cells and fibrotic response. Inflammation was assessed on a scale of 1 to 4 with a score of 1 indicating absence of inflammatory cells and a score of 4 indicating dense populations of lymphocytes and histiocytes. Fibrosis was scored on a scale of 1 to 4 with a score of 1 indicating no granular tissue and a score of 4 indicating dense granular tissue. Foreign body giant cells were evaluated on a scale of 1 to 4 with a score of 1 indicating no giant cells and a score of 4 indicating dense populations of giant cells. Non-parametric Kurskall-Wallis tests was conducted to evaluate statistical significance. SafraninO staining was conducted to evaluate the presence of cartilage.

### Results

No animals exhibited respiratory symptoms prior to surgery (Westley Score, 0). There were no intraoperative deaths or complications. Postoperative clinical Westley Scale scores, postmortem examination and overall duration of survival of the animals is detailed below in Table 1.

TABLE 1

| | | | | Survival |
|---|---|---|---|---|
| Pig | | | | Time, |
| No. | Wesley Score | Cause of Death Survival | | h:min |
| 1 | Zero | Early | Pneumothorax/ | 22:50 |
| | postoperatively | Death | Pneumo- | |
| | prior to drain | | mediastinum | |
| | displacement | | | |
| 2 | One (stridor with | Scheduled | Euthanized | 1050:03 |
| | agitation) through | Termination | | |
| | postoperative day 6 | | | |
| 3 | One (stridor with | Early | Euthanized | 380:03 |
| | agitation) through | Termination | secondary to | |
| | postoperative day 3 | | subcutaneous | |
| | | | abscess | |

Pig number one mechanically displaced the surgical Penrose drain overnight and died approximately 24 hours post-procedure, prior to displacement of the drain the animal had been clinical staple with Westley scores of zero. Necropsy that showed pneumothorax with pneumomediastinum as cause of death evidenced by collapsed lungs and connective tissue emphysema within the mediastinum, secondary to premature drain displacement. Pig number three had a Westley score of one through post-operative day three, which returned to zero thereafter. The third animal underwent early termination due to the development of subcutaneous abscess in the anterior soft tissue immediately deep to the surgical access site, but grossly distinct from the airway. The abscess did not involve or compress the airway. Animal number two had a Westley score of one through post-operative day 6 due to stridor with agitation, which returned to zero for the until it underwent scheduled termination six weeks post procedure. Necropsy showed a well-healed surgical site. Endoscopic exam of the trachea prior to termination showed well-healed subglottis and trachea without evidence of post-procedure stenosis. The posterior glottic mucosa overlying the implanted scaffold was pink to white without any gross abnormalities visible by line of sight from the endoscope.

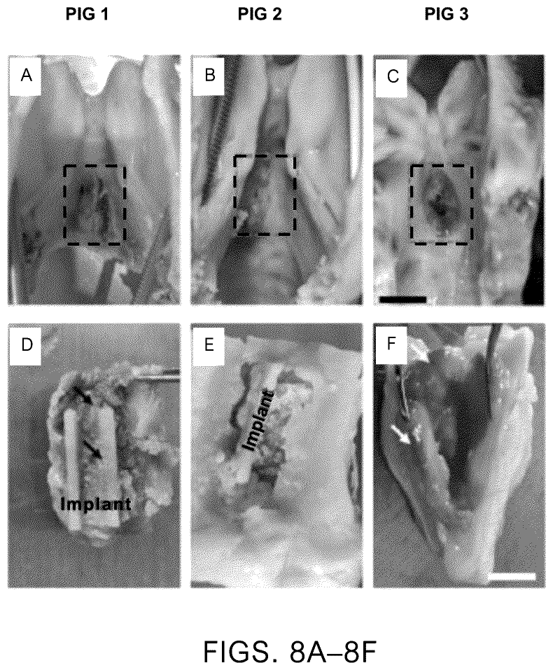

On evaluation of gross dissections of the larynx, there was evidence of progressive re-mucosalization in all three animals with the degree of mucosalization correlating to the implanted time. Grossly, pig one demonstrated signs of acute tissue inflammation, while pigs two and three showed signs of progressive mucosalization. Pig two, which underwent scheduled necropsy six weeks after surgical implantation, exhibited near complete re-mucosalization over the implant with minimal signs of inflammation. Two areas of raised scar similar to those seen via endoscopy confirm the proper section of the airway was captured during the endoscopic exam. Pig three exhibited hyper granulation tissue at the site of the incision through the posterior larynx with a persistent mucosal defect reflective of short post-operative time to termination. After further dissection, PCL scaffolds were exposed and showed connective tissue ingrowth as seen in FIGS. 8A-8F for each of pigs one to three (FIGS. 8A and 8D show pig one, FIGS. 8B and 8E show pig two, and FIGS. 8C and 8F show pig three).

On histologic examination, the rigid nature of the PCL scaffolds caused difficulties with sectioning and mounting. The PCL was displaced during the sectioning and appears as empty space on the stained slides. There was moderate fibrosis, inflammation and presence of foreign body giant cells in sections from pig 1 and pig 3 as anticipated from the gross tissue evaluation. Pig 3 exhibited acute inflammatory response at the early endpoint revealed moderate to high inflammation, granular tissue and presence of giant cells. SafraninO stain was used to assess cartilage ingrowth into the PCL scaffolds, but was limited due to the displacement of the PCL scaffolds during sectioning as mentioned above. Samples stained with hematoxylin and eosin were graded for fibrosis, inflammation, and foreign body giant cell response. There was significantly less inflammatory response, fibrosis and granulation in Pig 2 (p<0.01) as compared to pig 1 and pig 3. Implant from pig 2 also revealed cartilage in growth.

### Discussion

This preclinical pilot explores the early potential of a computer-aided designed and manufactured, 3D-printed, bioresorbable PCL posterior cricoid scaffold designed as an alternative to costal cartilage graft in the posterior cricoid split procedure. The extent to which the scaffolds met intended design criteria is as follows along with a discussion of potential benefits and future directions on further design optimization. This discussion is summarized in Table 2.

| Design Criteria | | Results |
|---|---|---|
| 1 | Eliminate need for cartilage | Posterior cricoid split performed |
| | harvest | successfully with 3D printed |
| | | scaffolds in three porcine models |
| 2 | Maximize airway cross | 3D printed scaffold design increased |
| | section | airway cross sectional area by an |
| | | additional ~39% compared to |
| | | traditional costal grafts |
| 3 | Scalable to several sizes | Series of different size 3D printed |
| | | scaffolds provide surgeons options to |
| | | promote finding the best fit for each |
| | | patient |
| 4 | Compressive strength | Scaffolds withstood compressive |
| | | load of expanded cricoid ring for up |
| | | to 44 days |
| 5 | Biocompatible material; no | Scaffolds are made of PCL, a |
| | future interventions | biocompatible, bio-absorbable |
| | | polymer that has been shown to |
| | | maintain structural integrity for |
| | | greater than 24 months |
| 6 | Minimize implanted material | Endoscopic and histologic evaluation |
| | and provide rapid | showed successful mucosalization in |
| | mucosalization | two porcine subjects |

With advancements in medical 3D printing and tissue engineering, translations of these technologies to clinical applications is rapidly expanding. With the proposed alternative approach to traditional costal cartilage reconstruction, several benefits are evident. Use of a scaffold-based solution eliminates the need for rib cartilage harvesting; our large animal study demonstrates early support that it is possible to use 3D-printed scaffolds prepared in accordance with certain aspects of the present disclosure in place of harvested tissue. Eliminating the need to harvest costal cartilage and associated potential complications, such as pneumothorax and costal harvesting site infections, is an obvious benefit. Moreover, having an alternative implant immediately available is likely to reduce operative time and anesthetic exposure. The average procedure time in our animal study was 101.7 minute. As envisioned, this approach could utilize a sizing “dummy” to select the ideal dimensions, then opening a specific implant, allowing for a patient specific fit.

The tools and techniques leveraged to design these posterior grafts allows for modifications that would otherwise not be feasible with traditional costal cartilage. The airway cross-sectional area of airway lumen can be optimized by imparting luminal concavity. This can theoretically increase the airway cross-sectional area by an additional 38.9% compared to traditional grafts based on calculations using a 5 mm scaffold (additional cross-sectional area of 0.187 cm²) and an average airway diameter in a 2-4 year old (0.48+/−0.08 cm³).

The scaffolds need to have sufficient strength to expand the posterior glottic aperture; our large animal study empirically shows that the PCL scaffolds compressive strength is adequate to withstand the compressive load of the expanded cricoid ring at time of procedure, immediately post-procedure and up to 44 days post-operatively.

This application is ideal for a material that has a slow bio-absorbable profile. By being biocompatible and bio-absorbable, future procedures for removal would not be necessary. The biomaterial used to construct the scaffolds in this example, PCL, is specifically chosen for its ability to maintain structural integrity for greater than 24 months in human clinical application and pre-clinical animal models. Furthermore, PCL material induces a lower inflammatory response than PLGA, as demonstrated by lower major histocompatibility index II and glial fibrillary acidic protein expression. The laser sintering process is able to rapidly fabricate implant devices with a defined external shape, internal pore size, and architecture, which promotes tissue ingrowth. The bioresorbable nature of the splint is intended to allow for growth of the native trachea while avoiding additional surgical and anesthetic exposures. Use of additional bioactive materials, such as hydrogel with growth factors, is believed to provide the capability to further increase the rate of cartilage ingrowth. Likewise, scaffold additives and coatings can promote decreased time to mucosalization.

The scaffold is refined in its design to minimize implanted material to encourage rapid mucosalization, as compared to a solid block of PCL, with the goal being to implant the minimal architecture needed to achieve airway expansion. Endoscopic exam and postmortem evaluation showed successful mucosalization in one of our subject animals (pig 2). Although pig 1 and pig 3 were terminated in advance, the post-mortem exams from these animals give a glimpse into the tissue response at different stages of post-operative healing (23 hr, 308 hr, and 1050 hr). In all three animals, there was no signs of graft instability on postmortem exams.

In conclusion, this early preclinical large animal pilot study details the multidisciplinary effort to produce and test a computer-aided designed, 3D printed, bioresorbable PCL posterior graft scaffold. The scaffolds offer possible reductions in intraoperative and postoperative complication, operative time reduction, and improved outcomes based on variation in scaffolds, which will allow patient specific optimization. In a Yorkshire porcine model, the scaffolds were found to be well tolerated based on post-operative Westley scores along with histological and endoscopic evaluation. The scaffolds were easily implemented and functioned as designed in this study offering proof of concept.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A tissue scaffold implant for expanding a stenotic lumen, the tissue scaffold implant comprising:
a bridge structure defining at least two angled sides;
a lateral anchor integrally formed with the bridge structure, wherein the lateral anchor is configured to be disposed against at least a portion of an external circumference of the stenotic lumen in a region near an opening formed in the stenotic lumen;
at least two seat regions defined between the at least two angled sides of the bridge structure and the lateral anchor, wherein the at least two seat regions are configured to be received within and support the opening within the stenotic lumen, and the bridge structure and lateral anchor comprise a bioacceptable material.

2. The tissue scaffold implant of claim 1, wherein the bridge structure comprises two wings defining the at least two angled sides and a concave junction is respectively defined between the lateral anchor and each of the two wings to define the at least two seat regions.

3. The tissue scaffold implant of claim 1, wherein an angle defined between each of the at least two angled sides of the bridge structure and the lateral anchor is less than or equal to about 120°.

4. The tissue scaffold implant of claim 1, wherein the bridge structure defines a trapezoidal pyramidal shape.

5. The tissue scaffold implant of claim 1, wherein the lateral anchor defines a solid body having at least one hollow central region.

6. The tissue scaffold implant of claim 1, wherein the lateral anchor defines a plurality of struts in a central region.

7. The tissue scaffold implant of claim 1, wherein the bridge structure comprises at least two solid walls defining each of the angled sides.

8. The tissue scaffold implant of claim 1, wherein the at least two angled sides each comprise a surface comprising at least one securement feature.

9. The tissue scaffold implant of claim 8, wherein the at least one securement feature comprises a plurality of angled support teeth spaced apart from one another.

10. The tissue scaffold implant of claim 1, wherein the at least one securement features is a textured surface, so that the at least two angled sides comprise the textured surface.

11. The tissue scaffold implant of claim 1, wherein the lateral anchor defines a curved sleeve that conforms to at least a portion of the exterior circumference of the stenotic lumen.

12. The tissue scaffold implant of claim 1, wherein the lateral anchor defines a rectangular shape.

13. The tissue scaffold implant of claim 1, wherein the lateral anchor defines a polygonal shape.

14. The tissue scaffold implant of claim 1, wherein the lateral anchor defines a truncated diamond shape and the bridge structure defines at least two wings that each define at least two angled sides complementary with the polygonal shape.

15. The tissue scaffold implant of claim 1, wherein the lateral anchor defines a solid body having an aperture defined therein configured to receive a cylindrical tissue graft.

16. The tissue scaffold implant of claim 14, wherein the tissue graft comprises an upper region of perichondrium and a lower region of cartilage.

17. The tissue scaffold implant of claim 1, wherein a cross-sectional shape of the tissue scaffold implant is a K-shape.

18. The tissue scaffold implant of claim 1, wherein the bioacceptable material is selected from the group consisting of: polycaprolactone (PCL), polyethylene glycol (PEG), polylactic acid (PLA), polyurethane (PU), polyglycerol dodecanedioate (PGD), extracellular tissue matrix, polysiloxane, nickel-titanium alloy (nitinol), polyetheretherketone (PEEK), polyetherketoneketone (PEKK), and combinations thereof.

19. The tissue scaffold implant of claim 1 further comprising at least one bioactive material.

20. The tissue scaffold implant of claim 1, further comprising one or more openings configured to receive a needle for creating a suture between the tissue scaffold implant and the stenotic lumen.
